(19) United States
(12) Patent Application Publication
(10) Pub. No.: US 2025/0179538 A1
(43) Pub. Date: Jun. 5, 2025

(54) APPARATUS AND METHOD FOR EXTRACTING BIOACTIVE COMPOUNDS FROM ETHANOL BREWERS STILLAGE OR FEED STOCK UTILIZED IN BIOCHEMICAL REACTORS

(71) Applicant: Immortazyme Company Ltd.

(72) Inventor: Vincent Yacyshyn, Calgary (CA)

(21) Appl. No.: 18/964,067

(22) Filed: Nov. 29, 2024

Related U.S. Application Data
(60) Provisional application No. 63/605,178, filed on Dec. 1, 2023.

(51) Int. Cl. C12P 7/40; C12N 9/26; C12N 9/50; C12P 7/04; C12P 13/00; C12P 17/02

(52) U.S. Cl. CPC C12P 7/40; C12N 9/2414; C12N 9/50; C12P 7/04; C12P 13/00; C12P 17/02

(57) ABSTRACT

Extracting bioactive compounds from stillage produced by brewing ethanol or feedstock utilized in a biochemical reactor.

### FIELD OF THE INVENTION

The invention relates to an apparatus and method for extracting bioactive compounds from ethanol brewers' stillage or feedstock utilized in biochemical reactors.

### BACKGROUND OF THE INVENTION

Although microscale methods have been used to increase the extraction power and batch capacity and improve sample quality, solvent amounts and extraction time compared to conventional extraction methods for various bio active compounds including polyphenols, commercial scale extraction has proven challenging. Conventional extraction of secondary metabolites is time-consuming and generates substantial amount of solvent waste. Zavala-Lopez. Plant Methods (2017) 13:81. DOI 10.1 186/s13007-017-0235-x.

There are numerous advantages and limitations of brewers spent grain extraction processes and the production of agricultural byproducts can provide new value added products from the brewers spent grain. Extraction methods have been applied and new techniques are needed for cost effective extraction. Bonifacio-Lopes et al. Crit Rev. Food Sci Nutr.2020; 60(16); 2730-2741.

It has been shown that byproducts of flaxseed oil extraction consisted of protein, mucilage, and polyphenol compounds. Extraction methods of the phenolic acids include dioxan/ethanol, water/acetone, water/methanol, and water/ethanol in addition to enzymatic hydrolysis. The addition of carbohydratases and/or proteases allowed for optimal results for removal of the flaxseed byproducts compared to conventional non enzymatic extraction. Dias Ribeiro et al. Hindawi Publishing Corporation. ISRN Biotechnology, Vol. 2013, Article ID 521067, 6 pages. Similarly, the industrial processing of raspberry pomace resulted in the lipophylic and hydrophilic phytochemicals. Various combinations of carbohydratases and proteases improved the extraction of these compounds. Saad et al. Journal of Food Science. 21 May 2019.10.1111/1750-3841.14625.

The kinetics of polyphenyl extraction from brewers spent grain using a batch system in the evaluation of antioxidant capacity of these extracts over time were evaluated to determine the optimal conditions of liquid/solid and ethanol/water solvent extractions were also studied with ultrasound assistance and this demonstrated the highest extraction rate in yield is well as the shortest extraction time. Patricelli's model proved the most suitable for describing extraction kinetics. Carciochi et al. Antioxidants 2018, 7, 45; doi:10.3390/antiox7040045.

Maceration has also showed the highest phenolic yield when applied to microwave extraction of brewer's spent grains. In this study, microwave assisted separation showed the phenolic compounds were simplified to monomers. Stefanello et al. Food Chemistry 239(2018) 385-401.

Polyphenols are secondary plant metabolites and represent a large and diverse group of substances abundantly present in fruits herbs and vegetables. Extraction methods including super critical fluid extraction highlight a promising ecofriendly alternative to extraction of polyphenols. The protective role of polyphenols against reactive oxygen, nitrogen species, UV light, plant pathogens, parasites, and preditors emphasizes their commercial value. The main problem in extracting these compounds is their low bioavailability and rapid metabolism. Mojzer et al. Molecules. 2016.21, 901,doi:10.3390/molecules21070901.

The value of polyphenols with regards to their antibacterial activity is well known. This activity depends on the lipophilicity and the electronic charge properties of the polyphenols. The antibacterial activity of most polyphenols depends on interactions between the polyphenols and bacterial cells. Bouarab-Chobane et al. Frontiers in Microbiology. 18 Apr. 2019, doi 10, 3389/fmicb.2019.00829.

Phytochemicals in corn bran and in corn bran fiber vary. The quantity of phytosterols in current corn bran fiber is approximately 1.0-11.3 mg per 100 g of fiber. Dapcevic-Hadnadev et al. in Sustainable Recovery and Reutilization of Cereal Processing By-Products.2018.

Phenolic compounds are parts of secondary metabolites found in many plant species that can be glycosides or aglycones, matrix free bound compounds, and polymerized or monomeric structures. The extraction process is challenging there are several conventional and unconventional techniques for extraction. Conventional extraction methods are mostly designed by utilizing larger volume of extraction solvents and manual procedures that are mostly dependent upon the feedstock. These methods include solid-liquid extraction or soxhlet extraction, liquid-liquid extraction and maceration. These conventional methods suffer many drawbacks. To overcome these challenges, additional methods include pressurized liquid extraction, subcritical water extraction, super critical fluid extraction, microwave assisted extraction, solid phase extraction, ultrasound extraction, hydrostatic pressure extraction, solid supported liquid-liquid extraction, matrix solid phase dispersion, and counter current chromatography. Additional improvements on conventional techniques include automation, enhanced selectivity, and reduce consumption of extraction solvents. Alara. Current Research in Food Science. 4(2021)22-214.

Brewers' spent grain is an abundant by-product rich in various bio active compounds. Extraction methods that have been attempted include super critical carbon dioxide, autohydrolysis, alkaline hydrolysis, solvent extraction, ultrasound assisted extraction, dilute acid hydrolysis, enzymatic hydrolysis, and microwave assisted extraction. There are advantages and limitations to each of these processes depending on the compounds to be harvested from the brewers spent grain. Bonifacio-Lopes. Critical Reviews in Food Science and Nutrition. 2020, Vol. 60, No. 16, 2730-2741.

The demand for polyphenols that are present in small amounts in many fruits vegetables and functional food is growing for many reasons. Extracting polyphenols is challenging because extraction techniques should not alter food quality Conventional techniques include Percolation, decoction, heat reflux extraction, Soxhlet extraction, and maceration. Advanced technologies for extraction include: ultrasound assisted extraction, microwave assisted extraction, super critical fluid extraction, high-voltage electrical discharge, pulse electric field extraction, and enzyme assisted extraction. Advanced techniques are 32 to 36% more efficient with approximately 15 times less energy consumption and produce higher quality extracts. Membrane separation and encapsulation are promising future techniques. Process parameters of significance include solvent type, solid and solvent ratio, temperature, and particle size. Sridhar et al. Environmental Chemistry Letters. (2021) 19:3400-3443.

The known methods for extraction in isolation of natural products has been reviewed in an article published by Zhang et al. Chin Med. (2018)13:20.doi.org/10.1186/s13020-018-0177-x. The extraction methods that have been identified include maceration, percolation, decoction, reflux extraction, Soxhlet extraction, pressurized liquid extraction, supercritical fluid extraction, ultrasound assisted extraction, microwave assisted extraction, pulsed electric field extraction, enzyme assisted extraction, and hydro distillation and steam distillation extraction.

Fat soluble nutraceuticals have been extracted from rice bran using subcritical di-methyl ether extraction. The combination of transesterification and subcritical di-methyl ether extraction was believed to be a simple 2 step method to extract and purify policosanol. Wongwaiwech et al. Nature Research. Scientific Reports.(2020) 10:21007.doi.org/10.1038/s41598-020-78011-z.

The effects of 3 factors: including pressure, temperature, and ethanol concentration were maximized to extract phenolic compounds from brewers spent grain with the use of supercritical carbon dioxide. A pressure of 35 mPA, 40° C. temperature, and 60% ethanol was felt to be optimum. The limitations of super critical carbon dioxide extraction were evident. Spinelli et al. J. of Supercritical Fluids 107(2016)69-74.

The purification of polyphenols from distiller's grains using macroporous resin has been evaluated. Phenolic content was analyzed by ultra performance liquid chromatography and tandem mass spectroscopy. Optimal conditions were noted with D 101 resin with a dosage of 3 g, 4 hours adsorption, and 3 hours desorption in an 60% ethanol eluent. A purification rate of 51% was noted. Polyphenolic compounds obtained included: epicatechin, ferulic acid, hydroxybenzoic acid, caffeic acid, syringic acid, quercetin, vanillic acid, and gallic acid. Wang et al. Molecules 2019. Apr. 2; 24(7); 1284.

The removal of phenol from aqueous solution has also been evaluated with nonfunctionalized hyper cross linked polymer and ion exchange resins. The extent of phenol adsorption is affected by pH. The desorption of the nonfunctionalized resin was achieved by 50% methanol to water with recovery of close to 90%. Caetano et al. Journal of Colloid and Interface Science 338(2009) 402-409.

Two effective ion exchange chromatography processes were studied to determine the separation and recovery of monosaccharides, organic acids, and phenolic compounds from two kinds of hydrothermal liquefaction hydrolysates. Anion and cation exchange resins were used in chromatography separation with recovery of 70-97%. Chen et al. Separation and Purification Technology. 172(2017)100-106.

U.S. Pat. No. 7,820,419B2 describes a process for producing a fermentation product within amylase and protease. This patent details use of protease to degrade proteins during the processing and production of fermentation products such as ethanol from starch containing material.

U.S. Pat. No. 9,057,087B2 is a method of producing a fermentation product comprising liquifying a starch containing material to dextrins in the presence of asparaginase to reduce Maillard products.

U.S. Pat. No. 7,820,419B2 relates to a process for producing a fermentation product from starch containing material. This material is treated with amylase and protease in the presence of a fermenting organism. The protease was used to enhance protein degradation and subsequently enhance oil production.

Canadian Patent 2815430C teaches compositions and methods for the production of fermentable sugars using genetically modified fungal organisms. This patent provides enzymes that find use in enhancing hydrolysis of cellulosic material to fermentable sugars. The patent is silent on the extraction of bio active compounds from this feedstock stock.

Russian Patent 2771261C2 describes a method for producing fermentation products from a starch containing material. The proteases used in this patent enhanced the breakdown of mash for subsequent oilseed production.

Chinese Patent 101138686A describes a method for extracting active ingredients from natural products and uses thereof. This patent teaches solid phase extraction of natural products including solid phase material as well as gas liquid solid phase processes and optimization of these processes but is silent with regards to distillers grains and subsequent products.

WO 2015164336A2 and WO 2016089816A1 are inventions that provide novel improved methods that allowed effective capture of valuable active ingredients in biomass. These patents teach extraction of 1 or more active ingredients from biomass using multiple solvents, alkaline aqueous extraction, evaporation and subsequent enzymatic hydrolysis into separate nucleic acids and biologic compounds. The invention is based on the discovery of novel and improved technologies that allow sufficient capture of valuable ingredients.

Russian Patent 2402242C2 describes the method for complex synthesis of a biologically active substances from alcohol wastes. This patent describes the separation of vinasse into liquid and solid fractions, filtering the liquid fraction, evaporating it, treating the concentrate with various organic solvents, filtering the precipitate, washing and drying and grinding subsequent precipitates, and then macerating this solution with ammonium oxalate and subsequently treating the concentrate with alcohol.

Fractionation of distillers dried grains with solubles by a combination of sieving and aspiration is a good source of protein and oil in animal feeds. High-protein and oil content is desired because of its high nutrition and economic value. Physical separation is an early approach to increase oil and fat content based on profiles of components. Fractionation of distillers dried grains with solubles by combination of sieving and aspiration. Food and bioproducts processing. Volume 103, May 2017, pages 76-85.

Extraction and valorization of the protein fraction from wheat based dried distiller's grain with solubles has been advocated. Methodologies have focused on analyzing nutritional value of animal feed. There is interest in valorization of the protein fraction into a range of potential products. Opportunities include extraction of individual amino acids with the potential to be transformed into building blocks for polymers or pharmaceuticals. N biotechnol. 2015. December 25th; 32 (6): 606-611.

US20170087596A1 published patent application describes a system and method for fractionated grain. A sieving apparatus for fractionating a grain product comprises a top chamber separated from the bottom chamber by a sieve; a top chamber cover defined by a pleuraltiy of openings that allow substantial vertical entry of an air stream into the top chamber when the interior of the sieving apparatus is under vacuum.

US20410242251A1 published patent application describes a system and method for stillage fractionation. Whole stillage undergoes a separation of its liquid portion from the solid portion. Devices include a screening centrifuge. The thin stillage may be provided to a 3 phase separator for separation.

US20220183318 published patent application describes a method whereby solids and liquids may be separated in a production facility. The process separates components in the process stream by using a mechanical device to separate the solids from the liquids based on a density difference. The process produces the liquids and solids, which may be further processed to create valuable animal feed products.

Advancing our understanding of bioreactors for industrial sized cell culture has been reviewed and understood as an opportunity to produce compounds or composition of value for biology and bioengineering. Traditionally, fuel ethanol biochemical reactors have not been viewed as a source of compounds or components for cell or protein culture. Advancing our understanding of bioreactors for industrial sized cell culture: Health care and cellular agriculture implications. American Journal of physiology-cell physiology. Volume 325. 3.

U.S. Pat. No. 6,673,532B2 is a patent describing bioreactor and bioprocessing techniques. This particular system relies on noninvasive optical chemical sensing technology where an optical excitation source excited an optical chemical sensor. The luminescence emitted from the chemical sensor for the amount of light observed by the chemical sensor is related to the concentration of analyte within the bioreactor.

W02011003615A2 discloses a perfusion bioreactor relating to continuous flow centrifugation of cells separated from a supernatant and recycled into a culture bag. The method is for culturing cells and combines with continuous flow centrifugation for separation of the medium from the cells.

U.S. Pat. No. 2,070,286A discloses a method of treating distillery slop. The invention is an improved method of treating distillery slops. The purpose of recovering the grain bearing material and utilizing such grain as a feed substance. Advances in treatment have been subject to fault as they require long periods of time to achieve material separation.

U.S. Pat. No. 1,426,457A discloses a process of recovering volatile organic acids from distillery waste.

U.S. Pat. No. 6,509,180B1 discloses a process for producing ethanol including a combination of biochemical and synthetic conversions resulting in high yield ethanol production and concurrent production of high value coproducts. Co products can include corn oil and high-protein animal feed containing the biomass produced in the fermentation.

Amongst the various compounds or compositions available for separation from a bioreactor included in a fuel ethanol plant include phenol. Phenols and phenolic acid could play a positive role in the treatment of infections caused by resistant bacteria since they have the abilities to link with and disable some bacterial enzymes essential for bacterial cell wall synthesis in a way which may modify the resistance of certain kinds of bacteria. A general overview of Phenols is included in the resource. General overview of phenolics from plant to laboratory, good antibacterial are not?Pharmacogn review 2017 Jul.-Dec.; 11 (22): 123-127.

There is a growing body of evidence that flavonoids show antibacterial activity against both gram positive and gram negative bacteria. The mechanisms of action of phenolic compounds on bacterial cells had been partially attributed to damage to the bacterial membrane, inhibition of purulence factors such as enzymes or toxin, and suppression of bacterial biofilm formation. What is more, some natural polyphenols, aside from direct antibacterial activity, exert a synergistic effect when combined with common chemotherapeutics. Many studies have proved that in synergy with antibiotics plant flavonoids pose a promising alternative for therapeutic strategy against drug resistant bacteria. International Journal environmental research and public health. 2018 October; 15 (10): 2321.

U.S. Pat. No. 2,550,254A discloses a process for extraction of antibiotic material. A process is described for preparing a powerful antibiotic substance which compromises the fruit pit of the tree Persea, treating said fruit pit with acetone at a temperature not substantially in excess of room temperature to form a non aqueous solution of the antibiotic material, separating the solution from the insoluble residue, and reducing the solution to dryness by subjecting the said solution to distillation at a reduced pressure to obtain a powerful antibiotic substance.

US20160273005A1 discloses a method for producing phenol from renewable resources by fermentation. The invention relates to a method of generating a recombinant host strain for producing phenol. The invention also provides the recombinant host strain for producing phenol as well as a method of producing phenol in said recombinant host strain.

U.S. Ser. No. 10/358,669B2 discloses an apparatus and method of reducing phenol levels in enzymatic solutions to enhance enzymatic activity in industrial bioprocessing. The method further relates to a method for extracting the phenols from a biological feedstock recovering and purified those extracted phenols.

An improved micro scale method for extraction of phenolic acids from maize has been described by using HPLC-DAD detection method. This improved extraction method increases the extraction power and batch capacity while reducing the sample quantity, solvent amount, and extraction time. It has also achieved a better replicability with a lower coefficient of variation than is possible with conventional extraction. An improved micro scale method for extraction of phenolic acids from maize. Plant methods (2017) 13:81.

### SUMMARY OF THE INVENTION

In a first embodiment, the present invention provides a safe, cost-effective separation method that is scalable and methodologically feasible in grain processing facilities. The separation method, in addition to being methodologically feasible, produces a unique combination of products that include bioactive compounds such as polyphenols. The unique characteristics of the final product meet criteria for various end users in a hereto for not described method. The process is simple and able to produce adequate quantities of bioactive compounds suitable for human or animal consumption in a methodologically robust manner.

In a second embodiment, the present invention provides a separation method of producing a compound or mixture of compounds from different fractions of feedstocks used in a bioreactor. The resultant compound or mixture of compounds is dependent on the bioreactor feedstock. The process allows for a compound or unique mixture of compounds to be extracted in a cost-effective manner based on the feedstock in use.

Previous attempts have been made to extract compounds or mixtures compounds from bioreactors. The present invention surprisingly takes feedstock that has been prepared with prior fractionation and allows for a simplified method of extraction of unique compounds or combination of compounds hereto for not described. The present process can include a step whereby pre-treatment of bran prior to the fermentation with alkali or alkaline hydrogen peroxide and subsequent acidification with ethanol precipitation with or without xylanase at higher temperature(s) to make corn fiber gum and remove (xylo)oligosaccharides and ferulic acid. Examples of the higher temperatures include greater than 80° C., preferably greater than 160° C. The higher temperature can exceed 200° C.

A preferred method of extraction may be used to alter valuable compounds or combinations of compounds as a unique signature of compounds derived from natural-based feedstocks not presently available.

Objectives of the first embodiment of the invention and other objectives can be obtained by a method of extracting bioactive compounds from ethanol brewers' stillage comprising:
providing stillage from an ethanol plant, the stillage comprising spent grains suspended in water;
adding a lyophilized enzyme to the stillage;
adding ethanol to the stillage to form a mixture of ethanol and stillage;
mixing the mixture of ethanol and stillage;
extracting the ethanol from the mixture of ethanol and stillage to form a solution of bioactive compounds dissolved in extracted ethanol and spent grains containing the lyophilized enzyme; and
separating the bioactive compounds from the extracted ethanol by distillation or evaporation of the ethanol.

Objectives of the second embodiment of the invention and other objectives can be obtained by a method of extracting bioactive compounds from different fractions of feedstock used in a biochemical reactor comprising:
extracting a plurality of feedstock fractions from different locations in the biochechemical reactor; and
extracting the bioactive compounds from at least one of the plurality of feedstock fractions.

A portable skid-based extraction apparatus can be utilized to extract the bioactive compounds.

### BRIEF DESCRIPTION OF THE DRAWINGS

FIG. 1 illustrates an ethanol brewing plant modified to include the present method of obtaining bioactive compounds from the ethanol stillage.

FIG. 2 illustrates a GC/MS spectrogram of the extract in the plant of FIG. 1.

FIG. 3 illustrates a GC/MS spectrogram of the extract using no enzyme in the plant of FIG. 1.

FIG. 4 illustrates a GC/MS spectrogram of the extract using a lyophilized enzyme in the plant of FIG. 1.

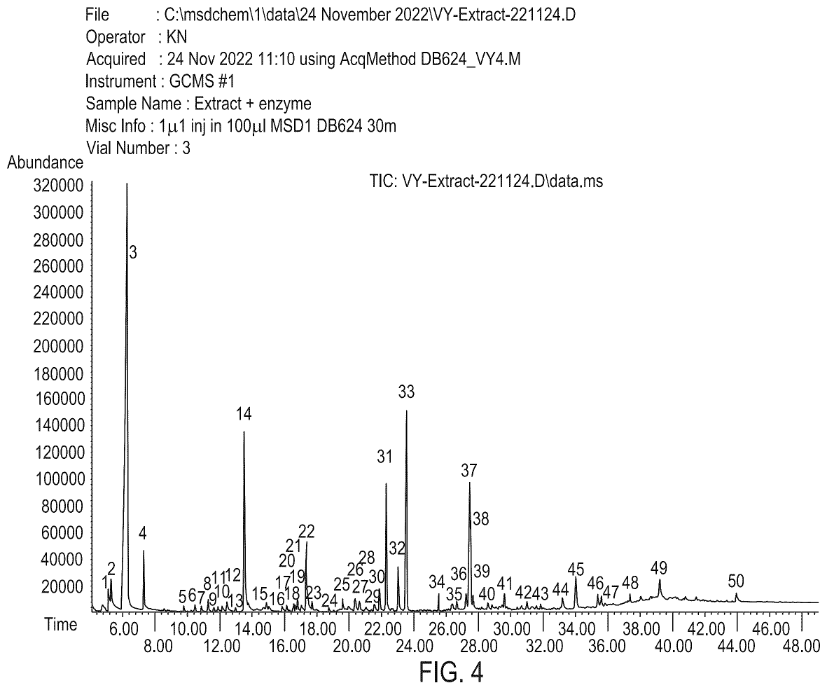

FIG. 5 illustrates a flowchart of an example of the extraction method.

FIG. 6 illustrates the test results of Sample 3 according to the second embodiment.

FIG. 7 illustrates the test results of Sample 4 according to the second embodiment.

FIG. 8 illustrates the test results of Sample 5 according to the second embodiment.

FIG. 9 illustrates the test results of Sample 6 according to the second embodiment.

### DETAILED DESCRIPTION OF THE INVENTION

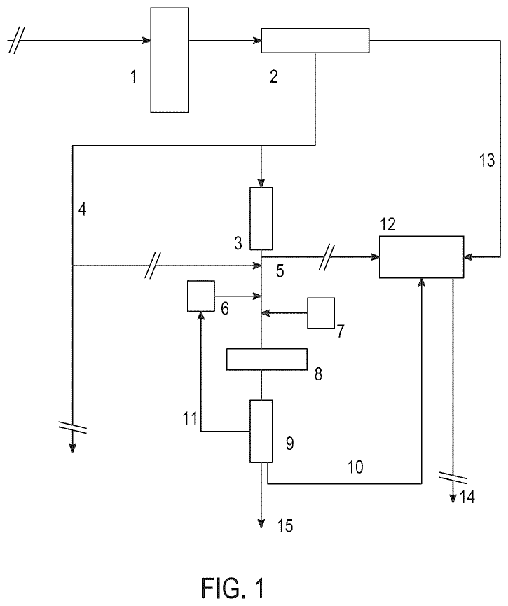

FIG. 1 illustrates a first embodiment of the present invention showing a modified biochemical reactor (ethanol plant). In the ethanol plant the feedstock is grains that are fermented to form a mixture of ethanol, water and spent grains. Fermentation products leave the Beer Well and pass through the stripping column 1 where ethanol and distiller's grains are separated to form stillage. The distiller's grains pass through the first centrifuge 2 where wet cake and thin stillage are separated. The thin stillage passes through a syrup evaporator 3 and a portion of the flow proceeds as thin stillage 4 and the syrup 5 may be added to the wet cake 13 and placed into the dryer 12. The product of this stream is distillers' dry grains with solubles 14.

The unique and novel process stream, takes the syrup from the syrup evaporator 3 and instead of placing the syrup into the dryer 12, the syrup is placed into a new process with the addition of ethanol 6 and lyophilized enzyme 7 to be mixed and separated in a second centrifuge 8. The liquid phase proceeds to an evaporator 9 and syrup 10 from the evaporator and solids from the second centrifuge 8 are recycled to the dryer 12. The ethanol 11 can recycled back into the ethanol stream for the new separation process. The bioactive compounds 15 are the precipitate or solids from the evaporator 9. Variable flow is indicated as “//” throughout the process.

The grain may be any combination of corn, wheat, or barley or other grains or combination thereof. This material is the feedstock for subsequent processing in a biorefinery. Examples of suitable sizes include 8 sieve (2.4 mm) to 100 (0.1 mm) sieve size grain, preferably 60 to 80 sieve grain.

An optional method is the creation of thin stillage with contents including distiller's grains. The distiller's grains are created by heating the previously ground grain to approximately 60 to 70° centigrade (although the temperature may be higher or lower). The subsequent mash is allowed to rest. The mash and effluent may be treated with any combination of lyophilized enzymes including amylase, glucoamylase, or proteases. Non-lyophilized enzymes should be avoided.

As this thin stillage flows through the biorefinery, the stream or portion of the stream of thin stillage and effluent may be subject to a wash with 80-99% ethanol. The thin stillage and effluent is washed with a minimum amount of ethanol in accordance with the flow rate of stillage and effluent through the biorefinery. The initial mixture of ethanol and thin stillage and effluent is centrifuged in a first centrifuge at 500 rpm for a retention time of 5 to 25 minutes, preferably 15 minutes.

Proteases may be added to the ethanol wash to enhance the efficacy of bioactive compound extraction.

After centrifugation at 500 rpm, the mixture in centrifuged in a first or second centrifuge at 1,000 to 10,000 rpm, preferably 10,000 rpm, for 5 to 15 minutes, preferably 10 minutes.

The thin stillage and effluent is subsequently washed with water and processed downstream into further distiller's grains products. The water washing is necessary to avoid potential combustion after ethanol washing.

The ethanol wash is evaporated preferably in a nonflammable inert gas but not limited to what is contained within a chamber (such as nitrogen) and the ethanol is recycled for additional treatment and washing of the feedstock. The concentration of ethanol used to wash the initial feedstock is minimized independent on the flow rate of thin stillage through the processing apparatus.

The precipitant from the evaporation of the ethanol is collected and no further purification is required. This precipitant can include any number of bioactive compounds suitable for human or animal consumption including but not limited to polyphenols (Cinnamic acid derivatives—ferulic acid and p-coumaric acid, Benzoic acid derivatives—caffeic acid, Anthocyanins-cyanidin-3-glucoside and delphinidin-3-glucoside, Flavones-tricin, and Lignans-secoisolariciresinol diglycoside). These compounds may also include: formic acid, propanol, propanone, propylene glycol, butanediol, furfural, furanmethanol, lactic acid, dihydroxyacetone,ethanol, oxirane, furancarboxylic acid, thiazole, maltol, furanone, pyranone, diglycerol, pentanoic acid, methylbutanoic anhydride, hydroxymethylfurfufral, benzenediol, succinic acid, tetraoxacyclododecanone, methylbutanoate, or benzoic acid.

The surprising and unique aspect of the present process is the simplicity, the use of lyophilized enzymes and avoidance of non-lyophilized enzymes. The ability to harvest a combination of bioactive compounds without over purifying them allows for a robust mechanical method of screening and extracting a portion of the bioactive compounds including polyphenols from the thin stillage. This unique process allows for a cost effective method of extracting bioactive compounds from thin stillage.

The biochemical reactor of FIG. 1 can also be utilized in the second embodiment with the reference numbers and description above applying to the second embodiment unless otherwise stated.

In the second embodiment of the invention, fractionated feedstock is extracted at various locations within the bioreactor processing stream using the desired extraction process. This is possible due to the unique compact simplicity and portability of the extraction skid coupled to the required combination of compounds requested by the end user. For example, traditionally compounds or combination of compounds may be extracted from syrup, wet cake, or distillers dry gains with solubles. With enhanced fractionation prior to extraction, compounds or combinations of compounds may be extracted from: stover, various fractions of fiber, syrup, clarified syrup, decanter products, and at least 3 different concentrated protein distiller's dry grain products.

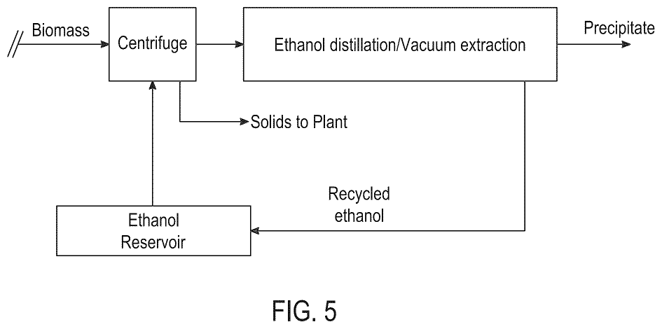

FIG. 5 shows a flow chart of an example of a suitable extraction process.

The unique desired compound or combination of compounds may be extracted at any number of processing locations within the feedstock stream or discharge of the bioreactor.

By pre-processing the feedstock and removing (xylo)oligosaccharides and ferulic acid, a skid-based extraction apparatus may be placed within the process stream to facilitate extraction.

Prior methods for extraction do not anticipate the simplicity and subsequent uniqueness and cost effectiveness of a skid based processing apparatus. The apparatus is composed of a solvent-based “washing” and “drying” apparatus.

The feedstock is preferably plumbed to a first centrifuge preferably at 10000 rotations per minute (rpm) or a relative centrifugal force of 12,500 g-Force equivalent with a residence time of 10 minutes at room temperature with a combination of ethanol at a ratio of ethanol to feedstock of 10:1. The centrifuge may operate preferably at 10,000 rpm (12,500 g-Force), but speeds of 5000 (4000 g-Force) to 10,000 rpm (12,500 g-Force), or even as low as 500 rpm (40 g-Force) to 5000 rpm (4000 g-Force) are feasible. Depending on the unique pre-processed feedstock, the ratio of ethanol to feedstock is preferably 10:1, but ratios of 5:1, or even as low as 1:1 are feasible.

The material discharged from the centrifuge is subsequently divided into 2 fractions. The solid material is reintroduced into the bioreactor feedstock or discharge processing stream. The liquid material is transferred to a distillation apparatus whereby the ethanol fraction is significantly diminished. The use of distillation may be preferentially enhanced with vacuum extraction.

The process may be performed as batch or continuous extraction.

The precipitate of the distillation is the unique compound or combination of compounds.

The ethanol fraction may be recycled for further use in the centrifuge.

What makes this present invention unique is that:
1) Enhanced preprocessing of the feedstock or discharge material, avoids extensive post processing of a solvent based reaction
2) Removal of (xylo)oligosaccharides and ferulic acid, in and of themselves, can be a product, but also alter the subsequent product availability.
3) The pre-processed feedstock or discharge allows for here to for unique compounds or combinations of compounds to be extracted.
4) The methods described significantly decrease the complexity of compound or combination of compound extraction in an unanticipated manner.
5) The operational robustness, scale ability, allow for cost effective production of compounds or combination of compounds here to for unanticipated.
6) The unanticipated simplicity allows for minimal waste of feedstock or discharge material.

### Examples of First Embodiment

### Example 1

310 g of number 60 sieve (0.250 mm) mixture of ⅓ corn, ⅓ barley, ⅓ wheat was added to 1.2 liters of water and heated to 70° Celsius (C). The mixture was allowed to rest for 90 minutes and the liquid decanted from the resultant distiller's grains.

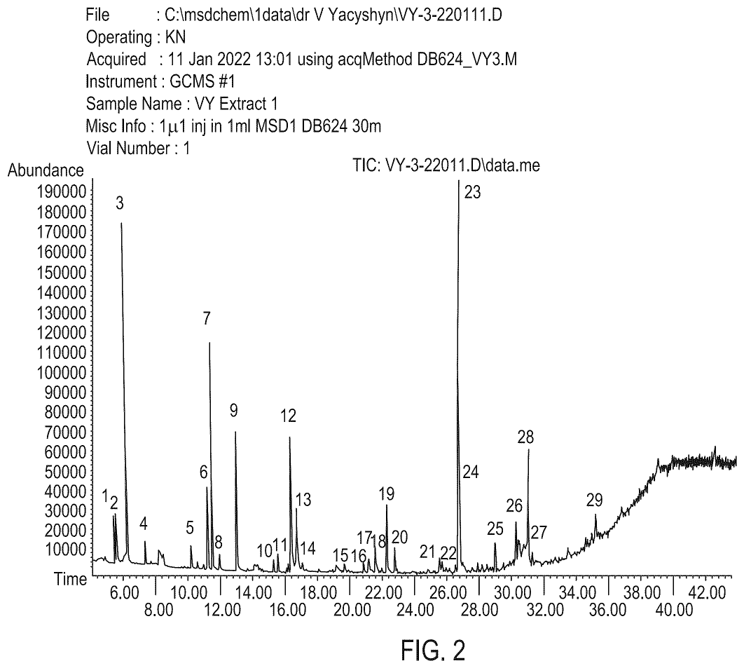

One (1.0) grams of the resultant distiller's grains were added to 10 mL of 80% ethanol. The sample was incubated at room temperature in a centrifuge at 500 rpm for 15 minutes and then centrifuged at 10,000 rpm for 10 minutes at room temperature. The ethanol was evaporated under a stream of nitrogen to 1 mL and 1 microL of the liquid phase was injected for gas chromatography/mass spectroscopy. NIST (United States National Measurement Institute) mass spectral library search was used to identify the peak of the resultant mass spectrum and compared to known standards. The 25th peak identified included phenolic compounds. See Table 1 and FIG. 2 GC/MS spectrogram.

TABLE 1

| Peak # | NIST Library Top Match | Quality |
|---|---|---|
| 1 | Formic acid | 56 |
| 2 | Acetaldehyde, hydroxy- | 49 |
| 3 | Acetic acid | 91 |
| 4 | 2-Propanone, 1-hydroxy- | 80 |
| 5 | 2-Propenoic acid, 2-hydroxyethyl ester | 83 |
| 6 | 3-Penten-2-one, 4-methyl- | 87 |
| 7 | 2-Propanone, 1-hydroxy- | 42 |
| 8 | Propanoic acid, 2-oxo-, methyl ester | 9 |
| 9 | Butanal, 3-hydroxy- | 32 |
| 10 | 2,3-Butanediol | 90 |
| 11 | Propanenitrile, 3-(1-methylethoxy)- | 78 |
| 12 | Furfural | 87 |
| 13 | Silane, ethyldimethyl- | 88 |
| 14 | 2-Furanmethanol | 95 |
| 15 | 4-Cyclopentene-1,3-dione | 64 |
| 16 | 1,2-Cyclopentanedione | 83 |
| 17 | 2-Cyclohexen-1-ol | 59 |
| 18 | 2-Furanmethanol, 5-methyl- | 90 |
| 19 | Butyrolactone | 80 |
| 20 | 2(5H)-Furanone | 87 |
| 21 | Isomaltol | 59 |
| 22 | Dihydroxyacetone | 74 |
| 23 | Pentanoic acid, 2,2-dimethyl-, ethenyl ester | 50 |
| 24 | 2H-Pyran-2,6(3H)-dione | 64 |
| 25 | Benzeneacetaldehyde | 87 |
| 26 | 2-Hydroxy-gamma-butyrolactone | 47 |
| 27 | Furaneol | 81 |
| 28 | Hydrouracil, 1-methyl- | 59 |
| 29 | Furyl hydroxymethyl ketone | 87 |
| 30 | 5-Hydroxymethyldihydrofuran-2-one | 45 |
| 31 | Maltol | 91 |
| 32 | 1,3-Propanediamine, N-methyl- | 72 |
| 33 | 4H-Pyran-4-one, 2,3-dihydro-3,5-dihydroxy-6-methyl- | 95 |
| 34 | 2-Imidazolidinethione | 43 |
| 35 | 1,2-Ethanediol, 1-(2-furanyl)- | 42 |
| 36 | (S)-5-Hydroxymethyl-2[5H]-furanone | 68 |
| 37 | 2(3H)-Furanone, dihydro-4-hydroxy- | 50 |
| 38 | 5-Hydroxymethylfurfural | 84 |
| 39 | Catechol | 81 |
| 40 | Butyl 2,5,8,11,14,17,20,23,26-nonaoxaoctacosan-28- | 42 |
| | oate | |
| 41 | Hydrazine, 1,1-diethyl-2-(1-methylpropyl)- | 53 |
| 42 | Ethanamine, 2-methoxy- | 43 |
| 43 | Ethanamine, 2-methoxy- | 53 |
| 44 | 1-(O-Hydroxyphenyl)-2-(methylamino)propane | 64 |
| 45 | 1,3-Propanediol, 2-ethyl-2-(hydroxymethyl)- | 37 |
| 46 | 1-[3-Hydroxypropyl]-aziridine | 43 |
| 47 | .beta.-D-Glucopyranose, 1,6-anhydro- | 49 |
| 48 | Pyrrolo[2,3-d]pyrimidin-4-one, 3-amino-7-[.beta.-d- | 48 |
| | ribofurasonyl]- | |
| 49 | 3-Deoxy-d-mannoic lactone | 27 |
| 50 | benzamide, N-[4-(heptyloxy)phenyl]-2,6-dihydroxy- | 35 |

### Example 2

310 g of number 60 sieve (0.250 mm) mixture of ⅓ corn, ⅓ barley, ⅓ wheat was added to 1.2 liters of water and heated to 70° C. The mixture was allowed to rest for 90 minutes and the liquid decanted from the resultant distiller's grains. 10 mL of distilled de-ionized water added to approximately 1 g maize (corn/barley/wheat mixture). Add 10 mL of Fortiva Revo* to each sample. 15 minute incubation at 25° C. at 500 rpm for each sample

10 minute centrifugation at 25° C. at 10,000 rpm for each sample Decant supernatant (approximately 10 mL water). Membrane separation of the supernatant of 60 to 80 sieve maize remnants (0.177 to 0.250 mm—regular filter paper). Add one vial (10 mL volume) of resin to each sample**. Incubate each sample 15 minutes at 25° C. in ultrasound bath. Incubate each sample 15 minutes at 25° C. at 500 rpm. 10 minute centrifugation of each sample at 25° C. at 10,000 rpm. Resuspend each resin pellet in 5 mL (2 mL) 95% ethanol. Incubate each sample 15 minutes at 25° C. in ultrasound bath. Incubate each sample 15 minutes at 25° C. at 500 rpm. 10 minute centrifugation of each sample at 25° C. at 10,000 rpm. Decant the supernatant of each sample for gas chromatography/mass spectroscopy. Evaporate the sample down to approximately 250 microL and inject 1 microL into the gas chromatography/mass spectrometer for analysis. NIST (United States National Measurement Institute) mass spectral library search was used to identify the peak of the resultant mass spectrum and compared to known standards. No gas chromatography/mass spectroscopy results were found in the resultant supernatant. Liquid enzyme did not allow for the extraction of bioactive compounds from the sample.
* Novozymes: Fortiva Revo. Liquid alpha-amylase and protease.
** Resin: Amberlite XAD-7. CAS 37380-43-1 MDL Number: MFCD00132705 AAL 1956422 L1956422

### Example 3

310 g of number 60 sieve (0.250 mm) mixture of ⅓ corn, ⅓ barley, ⅓ wheat was added to 1.2 liters of water and heated to 70° C. The mixture was allowed to rest for 90 minutes and the liquid decanted from the resultant distiller's grains.

### Sample No. 1.

10 mL of 95% ethanol added to 1 g maize (corn/barley/wheat mixture). 15 minute incubation at 25° C. at 500 rpm. 10 minute centrifugation at 25° C. at 10,000 rpm. Decant supernatant and store at −20° C. for analysis with gas chromatography/mass spectroscopy (supernatant is the free/solute phenolics).

### Sample No. 2.

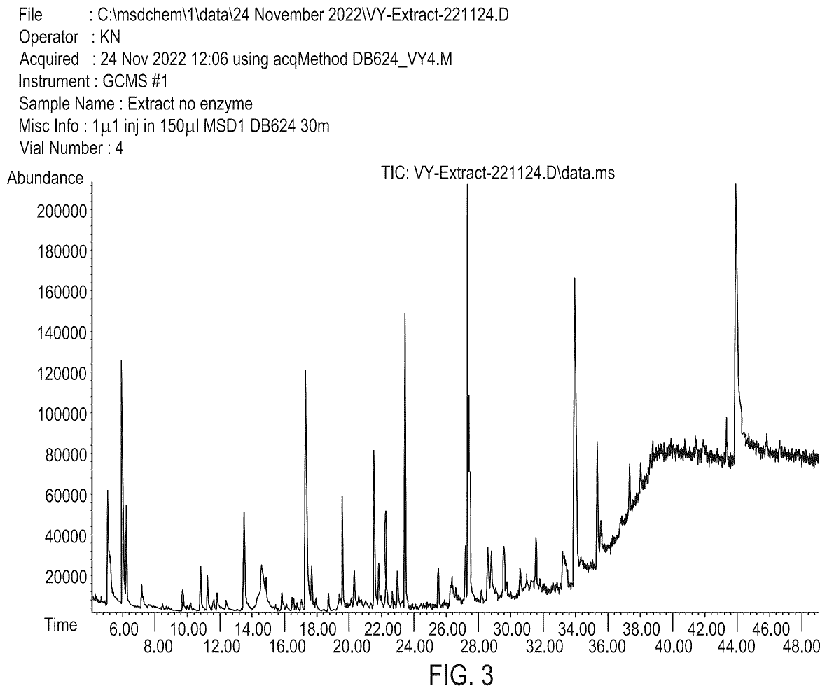

10 mL of 95% ethanol added to 1 g maize (corn/barley/wheat mixture). Add 25 mg (¼ vial) of lyophilized protease enzyme*. 15 minute incubation at 25° C. at 500 rpm. 10 minute centrifugation at 25° C. at 10,000 rpm. Decant supernatant and store at −20° C. for analysis with gas chromatography/mass spectroscopy. (supernatant is the free/solute and possibly lipophilic phenolics). Decant the supernatant of each sample for gas chromatography/mass spectroscopy. Evaporate the sample down to approximately 250 microL and inject 1 microL into the gas chromatography/mass spectrometer for analysis. NIST (United States National Measurement Institute) mass spectral library search was used to identify the peak of the resultant mass spectrum and compared to known standards. See GC/MS spectrogram FIGS. 3 (extract no enzyme) and 4 (extract with enzyme) respectively. Lyophilized enzyme (powdered) surprisingly and unexpectedly did not have added liquid contaminants or interfere with the extraction of bioactive compounds.
* Millipore Sigma ALCALASE enzyme, Bacillus lichenformis. Catalogue #126741500 ML.

### Examples of the Second Embodiment

### Example 4

### Sample Preparation

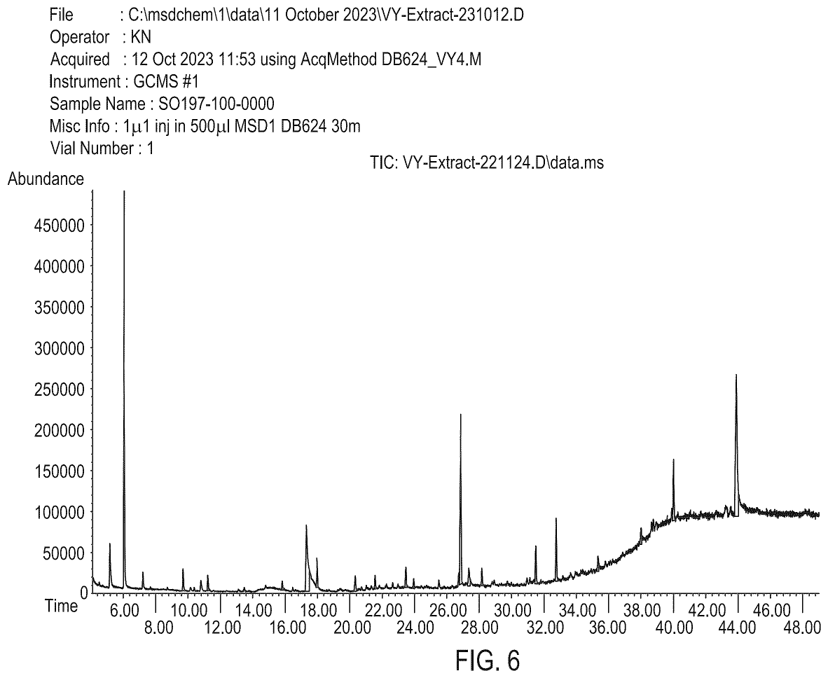

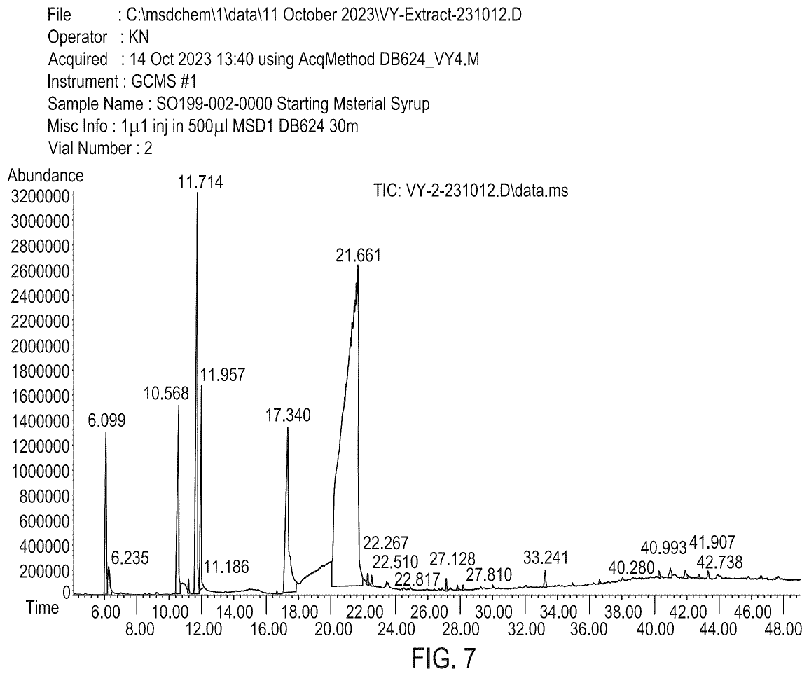

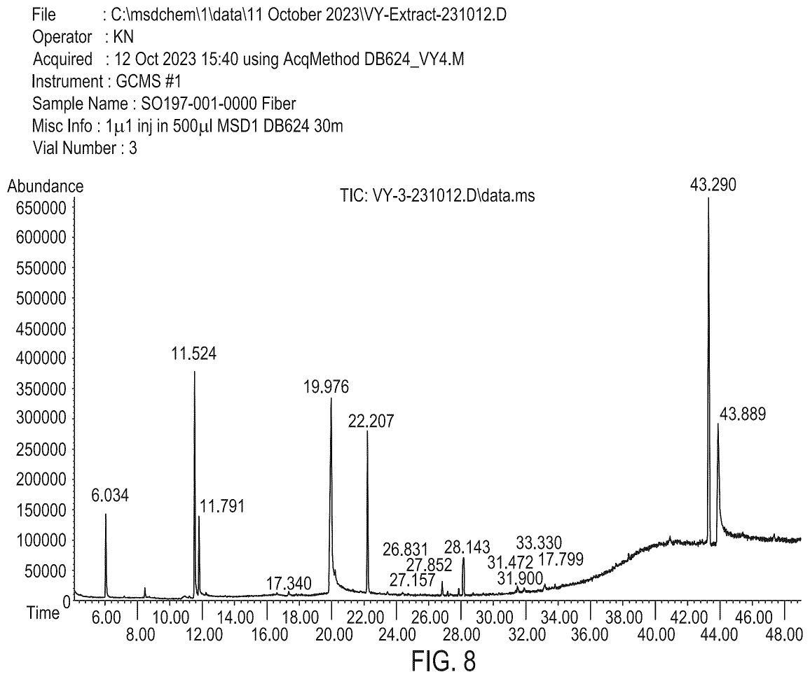

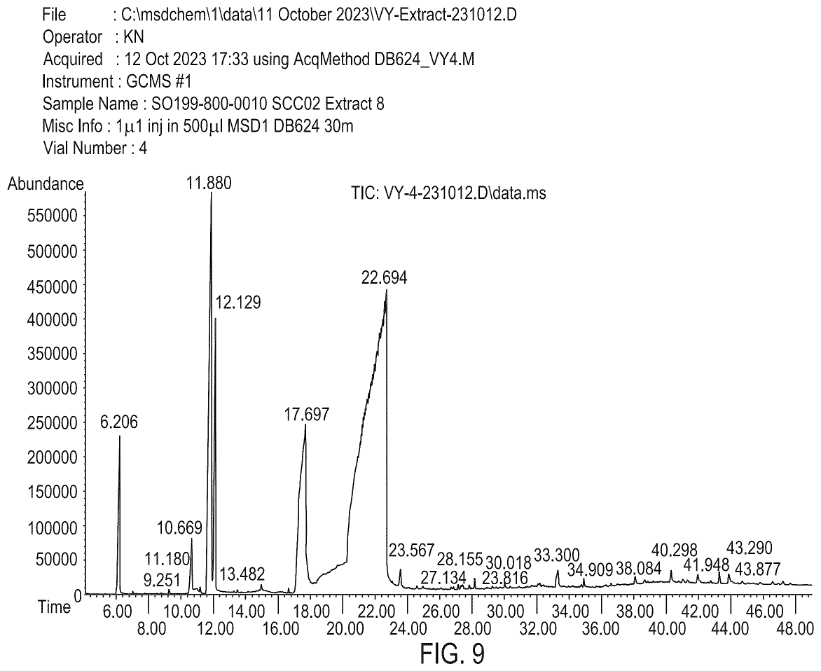

10 mL of 95% ethanol added to 1 g maize (distillers' grain*)
15 minute incubation at 25 degree c. at 500 rpm
10 minute centrifugation at 4 degree c. at 10,000 rpm**
Decant supernatant and store at −20 degree c. for analysis with gas chromatography/mass spectroscopy.
(supernatant is the free/solute phenolics)
*The Distillers' grain samples are: Syrup, ICMB Decanter, High Pro DDG, Gen 1.5 DDG, Protomax, lCMB Fiber, TS4 Solmax, S0197-100-0000 Stover, S0199-002-0000 Starting Material-Clarified Syrup, SO197-001-000 Fiber, and SO199-800-0010SCCO2 Extract #8.
   Sample 3 results are below and shown in FIG. 6.
Sample SO197-100-0000 Stover
1. Acetaldehyde, hydroxy
2. Acetic acid
3. 2-Propanone, 1-hydroxy-
4. 2-Propenoic acid, 2-hydroxyethyl ester
5. Propanoic acid, 2-oxo-, methyl estyer
6. 2-Cyclopenten-1-one, 2-hydroxy-
7. 1,2-Ethanediol
8. Hexanoic acid
9. 2-Hydroxy-gamma-butyrolactone
10.4,5 Diamino-2-hydroxypyrimidine
11.4H-Pyran-4-one,2,3 dihydro-3,5-dihydroxy-
12. Octanoic acid
13. Benzofuran, 2,3-dihydro-
14. DL-3-Aminoisobutyric acid-N-Dimethylamino
15.2-Methoxy-4-vinylphenol
16. Vanillin
17. Benzaldehyde, 4-hydroxy-
18.7-Methy-2-phenyll H-indole
19. Anthrancene, 9,10-dihydro-9,9,10-trimethyl-
20. Trans-4-Dimethylamino-4-methoxychalcone
21. N-Hexadecanoic acid
   Sample 4 results are shown below and in FIG. 7.
Sample SO 199-002-0000 Starting Material Syrup
1. Acetic acid
2. Acetic acid
3. Propylene glycol
4. Propanoic acid, 2-hydroxy-, ethyl ester
5. 2,3-Butanediol
6. 2,3-Butanediol
7. L-Lactic acid
8. Glycerin
9. Phenylethyl Alcohol
10.2-Pyrrolidinone
11. Divinyl sulfide
12. Thiophene,2,3-dihydro-
13.2-Methoxy-4-vinylphenol
14. Benzeneethanol, 4-hyroxy-
15.2-(Acetoxymethyl)-3-(methoxycarbonyl)biphenylene
16. 1-Butylpyrrolidine
17.2-(2-Amino-benzoimidazol-1-yl)-1-(4-methyoxy-phenyl)-ethanone
18. Benzo(h)quinoline,2,4-dimethyl-
   Sample 5 results are shown below and in FIG. 8.
Sample SO197-001-0000 Fiber
1. Acetic acid
2. Hydrazine, (2-methylpropyl)-
3. 2,3-Butanediol
4. 2,3-Butanediol
5. (3-Methyl-oxiran-2-yl)-methanol
6. Glycerin
7. Phenylethyl Alcohol
8. Benzofuran,2,3-dihydro-
9. Diglycerol
10.2,4-Decadienol
11.2-Methoxy-4-vinylphenol
12. Vanillin
13. 1,2,3-Thiadiazole-4-carboxamide,N-(2,6-dichlorophenyl)
14. N-m-Tolyl-succinamic acid
15.4-Phenyl-3,4-dihydroisoquinoline
16. Hexadecanoic acid, ehthyl ester
17. N-Hexadecanoic acid
   Sample 6 results are shown below and in FIG. 9
Sample SO 199-800-0010 Extract #8
1. Acetic acid
2. 2,4,5-Trichlorophenyl cinnamate
3. Propylene glycol
4. Propane, 1-(1-ethoxyethoxy)-
5. 2,3-Butanediol
6. 2,3-Butanediol
7. 2-Octanol
8. L-Lactic acid
9. Glycerin
10.4H-Pyran-4-one2,3-duhydro-3,5-dihydroxy-6-methyl-
11. Divinyl sulfide
12. N-Aminopyrrolidine
13.2-Methoxy-4-vinylphenol
14. Carboninc acid, neopentyl 4-cyanophenyl ester
15. Benzeneethanol, 4-hydroxy-
16.2,3,5,6-Tetrafluoroanisole
17. 2-(4-Methyl-piperidin-1-yl)-N-naphthalen-1-yl-acetamide
18. Beta-(4-hydroxy-3-methoxyphenyl)proprioninc acid
19. Adenine
20. Hexadecanoic acid, ethyl ester
21. N-Hexadecanoic acid

### REFERENCE NUMBERS

1 Stripping column
2 First centrifuge
3 Syrup evaporator
4 Thin stillage
5 Syrup
6 Ethanol
7 Lyophilized enzyme
8 Second centrifuge
9 Evaporator
10 Syrup
11 Ethanol
12 Dryer
13 Wet cake
14 Distillers' dry grains with solubles
15 Bioactive compounds
// Variable flow

While the claimed invention has been described in detail and with reference to specific embodiments thereof, it will be apparent to one of ordinary skill in the art that various changes and modifications can be made to the claimed invention without departing from the spirit and scope thereof.

1. A method of extracting bioactive compounds from feedstock utilized in a biochemical reactor comprising:
extracting a plurality of feedstock fractions from different locations in the biochechemical reactor; and
extracting the bioactive compounds from at least one of the plurality of feedstock fractions.

2. The method according to claim 1, further comprising
adding a lyophilized enzyme to the at least one of the plurality of feedstock fractions to form a feedstock mixture;
adding ethanol to the feedstock mixture;
extracting the ethanol from the feedstock mixture to form a solution of bioactive compounds dissolved in extracted ethanol and feedstock containing the lyophilized enzyme; and
separating the bioactive compounds from the extracted ethanol by distillation or evaporation of the ethanol.

3. The method according to claim 1, wherein the bioactive compounds comprise a polyphenol.

4. The method according to claim 3, wherein the polyphenol comprises at least one selected from the group of cinnamic acid derivatives-ferulic acid and p-coumaric acid, Benzoic acid derivatives—caffeic acid, Anthocyanins-cyanidin-3-glucoside and delphinidin-3-glucoside, Flavones-tricin, and Lignans-secoisolariciresinol diglycoside.

5. The method according to claim 1, wherein the bioactive compounds comprise at least one of formic acid, propanol, propanone, propylene glycol, butanediol, furfural, furanmethanol, lactic acid, dihydroxyacetone, ethanol, oxirane, furancarboxylic acid, thiazole, maltol, furanone, pyranone, diglycerol, pentanoic acid, methylbutanoic anhydride, hydroxymethylfurfufral, benzenediol, succinic acid, tetraoxacyclododecanone, methylbutanoate, or benzoic acid.

6. The method according to claim 1, further comprising using a skid-based extraction apparatus for extracting the plurality of feedstock fractions from different locations in the biochechemical reactor and extracting the bioactive compounds from at least one of the plurality of feedstock fractions.

7. The method of claim 1, further comprising pre-treatment of the feedstock prior to fermentation of the feedstock, the pre-treatment comprising treating the feedstock with alkali or alkaline hydrogen peroxide, subsequent acidification followed by an ethanol precipitation with or without xylanase at higher temperature(s) to produce corn fiber gum and removing (xylo)oligosaccharides and ferulic acid to alter downstream products.

8. The method according to claim 7, wherein the higher temperature(s) is greater than 80° C.

9. The method according to claim 7, wherein the higher temperature(s) is greater than 160° C.

10. The method according to claim 7, wherein the higher temperature(s) is greater than 200° C.
